(19) United States
(12) Patent Application Publication
(10) Pub. No.: US 2006/0277801 A1
(43) Pub. Date: Dec. 14, 2006

(54) Insole

(76) Inventors: Werner Schwarze, Jena (DE); Rolf Radel, Herne (DE)

Correspondence Address: Pandiscio & Pandiscio, 470 Totten Pond Road, Waltham, MA, 02451, US

(21) Appl. No.: 11/449,968

(22) Filed: Jun. 9, 2006

(30) Foreign Application Priority Data
Jun. 10, 2005 (EP) 05012477.5

(51) Int. Cl. A43B 13/38; A61F 5/14; A43B 23/00

(52) U.S. Cl. 36/141; 36/153; 36/43

(57) ABSTRACT

In an insole (1) for the massage or orthopedic treatment of human feet (2) that is arranged in a shoe (3), it should be possible completely to remove the bacteria, fungus and fungal spores transferred to the insole without thereby impairing the shape or the functions of the insole (1).

This is achieved in that the insole (1) consists of a thermoplastic elastomer material and that the material is heat-resistant to such an extent that insole can be sterilized using hot steam or pressurized hot steam.

The present invention relates to an insole for the massage or orthopedic treatment of human feet that is arranged in a shoe, and to a process for manufacturing an insole in accordance with the pre-characterizing clause of patent claim 11.

An insole of this type can be seen, for example, in EP 0 917 835 B1, and consists of a first soft lower layer of non-allergenic rubber material and a second thin upper layer of material that is soft to the touch and is suitable for contact with the sole of the foot. Several hollow cells are formed between the two layers. The hollow cells are filled with individual elastic particles, most of which have an irregular shape, that are made from a non-allergenic rubber, so that these form elastic convexities with a thickness and elasticity that are suitable for stimulating the reflex zone of the proprioceptors and baroreceptors as well as the reflex zone points in the sole of the foot.

DE 36 27 538 A1 describes an inlay for shoes, in particular an insole, comprising a reflex zone grid of spacers that form cells. The reflex zone grid is attached to a basic mould for the sole of a foot and consists of a foamed material such as PUR form or also latex. The reflex zone grid is covered by an upper sole layer that is air-permeable, compatible with the skin and has active breathing properties. It should be possible to insert medicinal herbs and medicinal mineral particles into the individual cells of the insole so that they should come into contact with the sole of the foot.

It has proven to be a disadvantage of the state-of-the-art referred to that the insoles constructed in this way become contaminated within a short period of time and this contamination cannot be removed from the insole without the functions and/or shape of the insole being impaired.

However, because insoles of this type should be used for diabetics, for example, with open foot sores due to the increased sugar load in their body, in particular in the soles of their feet, the insoles not only become dirty but are also contaminated with bacteria or fungus.

The insoles should not only perform the function of massaging the reflex zones in the sole of the human foot and promoting blood flow through these zones, but should also possess orthopedic properties under certain circumstances, for example in order to allow them to be used for treating abnormal positions or abnormal posture caused by muscular dysbalances or static deformation of the bones, therefore the insoles should be worn by the patient on a regular basis. Permanent use of this type also leads to contamination and infection of the insole with bacteria, fungus and fungal spores because the insole is located in an enclosed space for protracted periods, and this space is heated by the human foot. Often, this leads to perspiration and increased deposits that have an effect on the insole and accrete within it. The selected material properties of prior art mean that insoles structured in this way cannot be sterilized by steam pressure or by a certain degree of heat treatment, since this would irreparably damage this insole of prior art.

It is therefore the function of the present invention to provide an insole of the aforementioned type by means of which the foot can be massaged or orthopedically supported in a suitable way, and also to make it possible to sterilize this insole without thereby losing the massage or orthopedic support properties of the insole or the insole losing its shape and function as a result of the sterilization by steam pressure or heat.

This function is achieved in the present invention in that the insole consists of a thermoplastic elastomer material and that the material is heat-resistant to such an extent that the insole can be sterilized by hot steam or pressurized steam.

In order for the insole configured in accordance with the present invention to effect its massage and/or orthopedic treatment method, the insole is formed with a contact layer facing towards the foot and a support layer facing towards the shoe. The contact layer and the support layer are in this case firmly connected to one another in their edge zones, and one or more hollow cells are formed in between them by means of quilting seams or bonding seams or by means of a glue that separate or separates the hollow cells. Massage or support elements can be inserted in one or more of the hollow cells in order to stimulate the reflex zones of the sole of the foot and/or to provide orthopedic support for the sole of the foot.

It is beneficial for the massage or support elements to have a rotationally symmetrical shape, and in a preferred embodiment, to be cylindrical in shape and for them to be manufactured from a thermoplastic elastomer. Depending on the individual treatment method, it is also possible for one or multiple-piece cushions to be inserted that consist of a jacket which can be filled with the massage or support elements, the gel or foam or air, in which case the jacket of the cushions, the gel or the foam consist of a thermoplastic material.

It is also possible for only one hollow cell to be provided in between the contact layer and the support layer and for a one-piece massage or support element to be arranged within this hollow cell in order to massage the foot or provide it with orthopedic support.

The process for manufacturing a two-layer insole is characterized by the following process steps:
Working a slit-shaped opening into one of the two layers of the insole so that one or more of the hollow cells can be accessed from outside,
Filling a plurality of massage or support elements through each of the worked-in openings,
Alternatively, inserting at least one cushion into the particular hollow cell, the cushion being filled with the massage or support elements, with gel, with foam or with air, and
Closing the openings

Further advantageous embodiments of the invention are derived from the subordinate claims.

The material used for the insole and its layers, and the material for the massage and support elements, is the same material and the material is heat-resistant and does not become deformed even during a sterilization process using steam or steam pressure, but rather retains its original shape and elasticity, therefore the entire insole can be sterilized at low cost and any number of times. As a result, contamination and dirt resulting from bacteria, fungus or fungal spores can be easily removed without having to renew the insole and without its shape and functions being impaired by the sterilization.

This results in increased wearing comfort, because the sole of the foot is no longer continuously exposed to poor hygiene. Also, the illnesses which frequently occur with open foot sores in particular are reduced by the sterilization of the insole.

At the same time, the insole retains its massage or support function with the effect that the reflex points of the sole of the foot are acted upon in the usual way.

The drawing shows four embodiments of an insole configured in accordance with the present invention, the details of which are explained below. In the drawing,

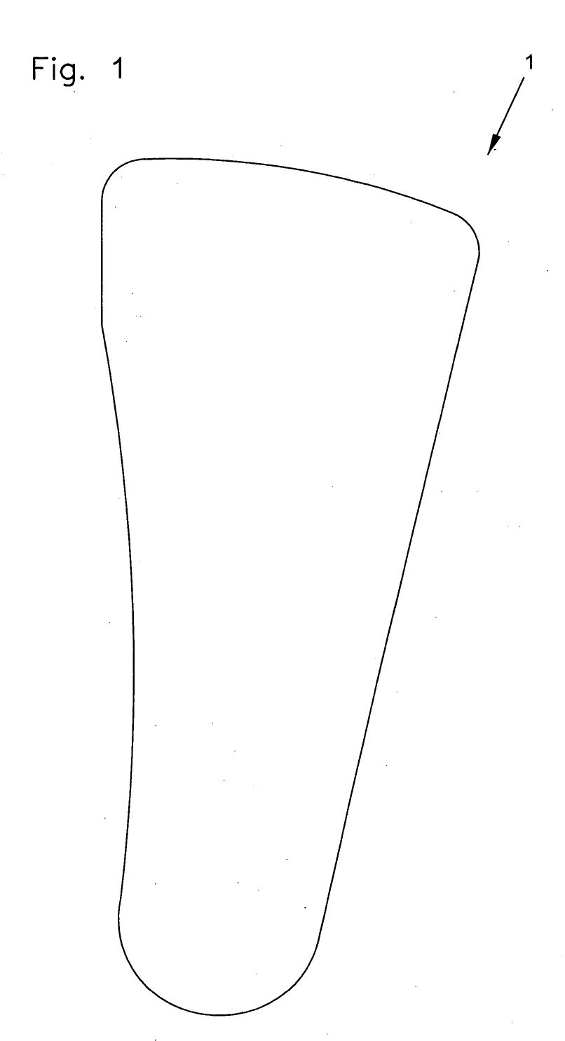

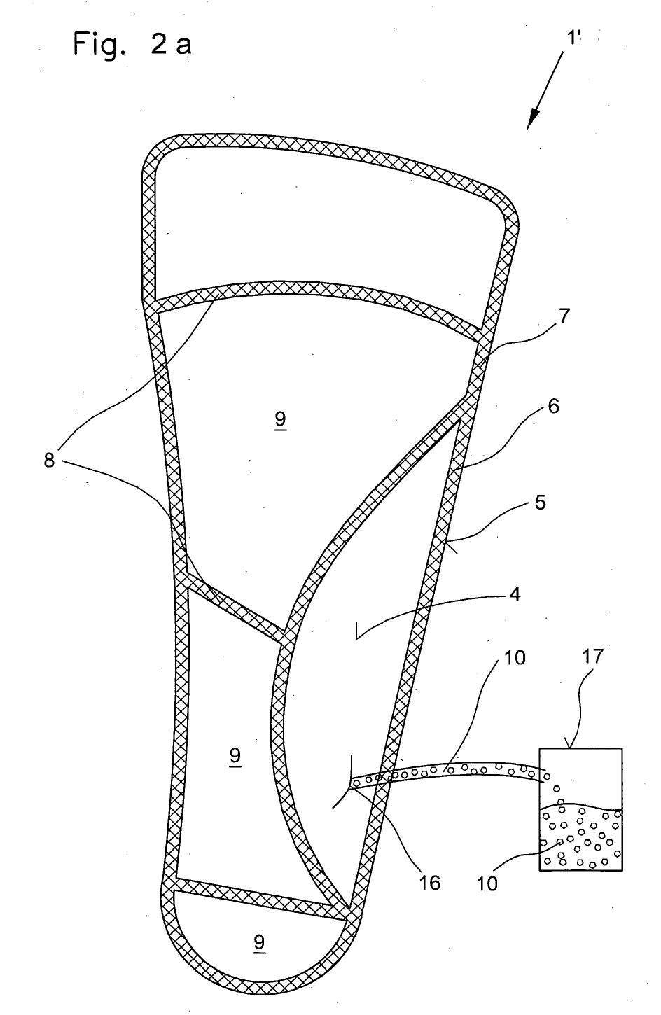

FIG. 1 shows a first sample embodiment of a single-layer insole, in a plan view,

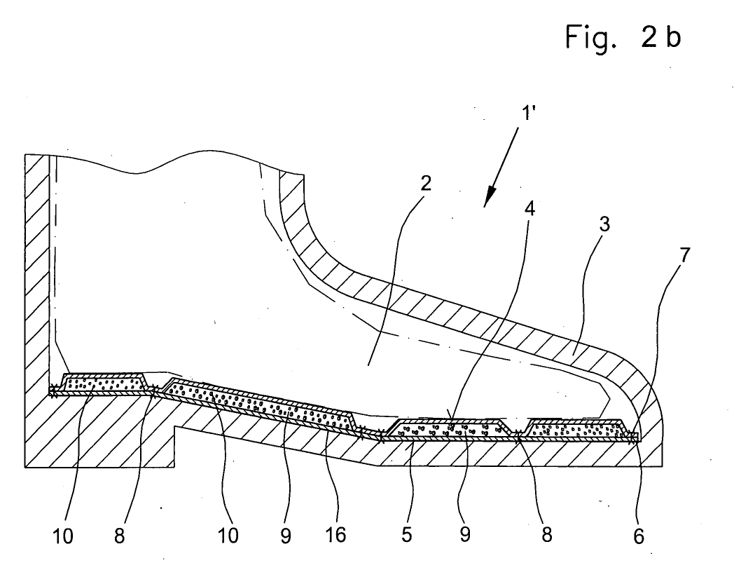

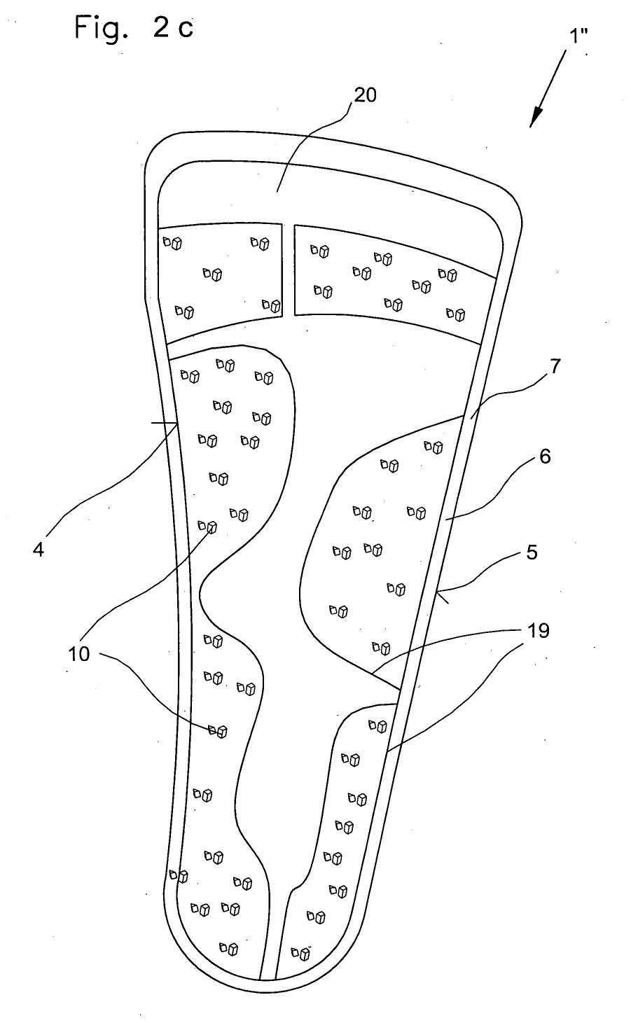

FIG. 2a shows a second sample embodiment of a double-layer insole with several hollow cells separated from one another by quilting seams, in a plan view,

FIG. 2b shows the insole in accordance with FIG. 2a along the section II-II in a shoe,

FIG. 2c shows the insole in accordance with FIG. 2a in another sample embodiment,

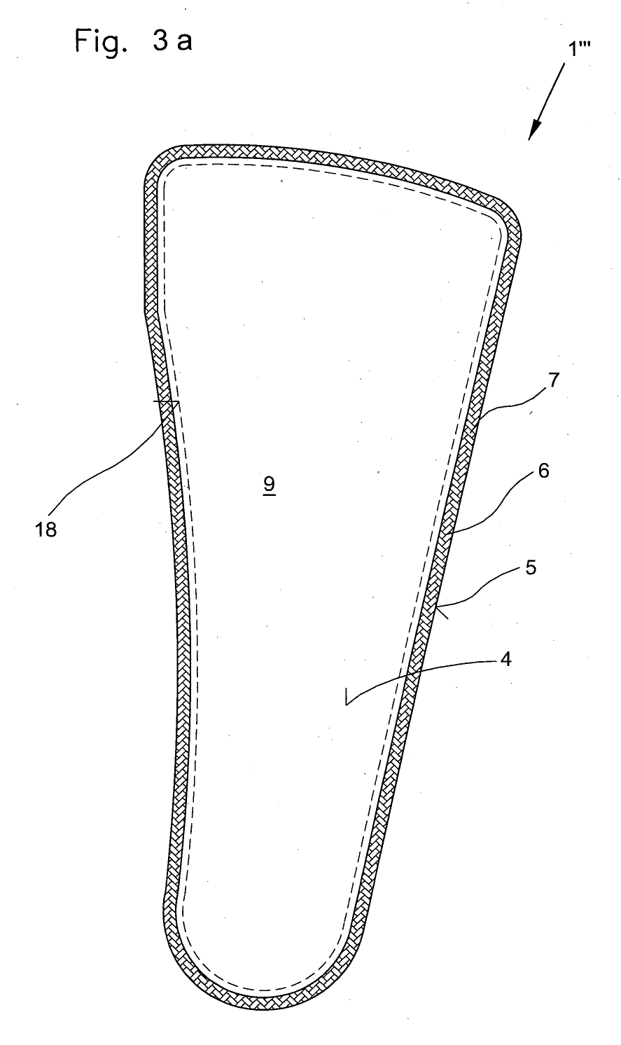

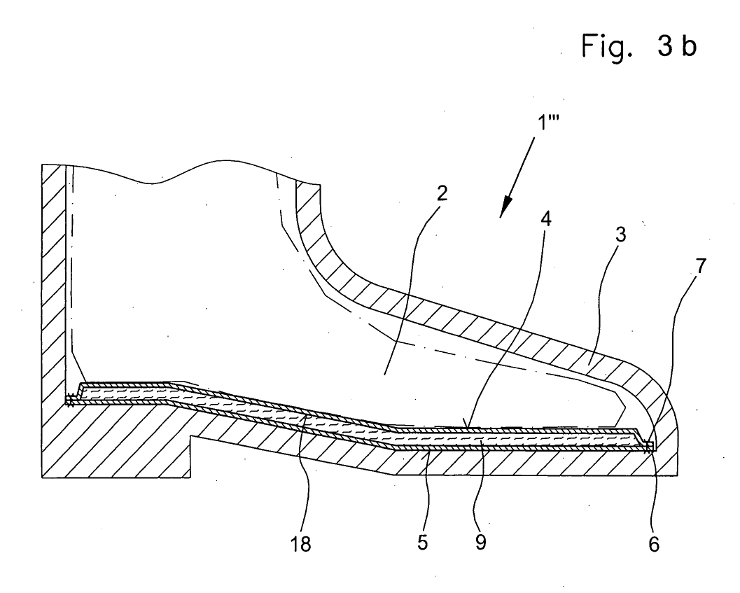

FIG. 3a shows a third sample embodiment of a double-layer insole in which a plate is inserted, in a plan view,

FIG. 3b shows the insole in accordance with FIG. 3a along the section III-III in a shoe and

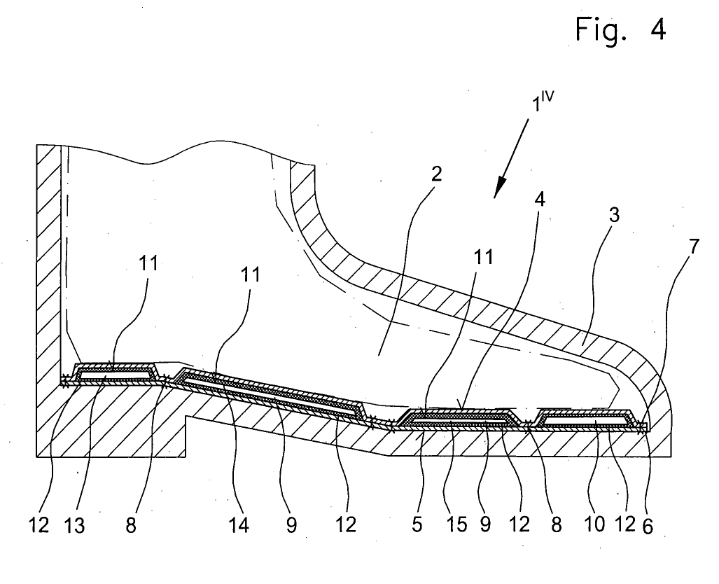

FIG. 4 shows a fourth sample embodiment of a double-layer insole with several hollow cells separated from one another by quilting seams, into each of which a cushion is inserted, in a side view.

FIG. 1 shows a single-layer insole 1 consisting of a thermoplastic elastomer material that can be sterilized by means of steam or steam pressure, for example. The shape of the insole 1 is modeled on that of a human foot and the insole 1 can be inserted in shoes so that the perspiration occurring inside the shoe is absorbed by the insole 1 and can be washed out of this by sterilization without the shape or the functions of the insole 1 being changed.

FIGS. 2a and 2b show that the insole 1 is arranged in a shoe 3 and that the insole 1 massages a foot 2 inserted in the shoe 3 on the known reflex zone points of the sole of the foot or that the foot 2 is orthopedically supported by the insole 1.

For this purpose, the insole 1 has a double-layered structure and consists of a contact layer 4 facing the foot 2 and a support layer 5 in contact with the inside of the shoe 3. The two edge zones 6 of the contact layer 4 and the support layer 5 are firmly connected together by means of a circumferential seam 7 and have a shape that is chiefly modeled on the external contour of the human foot 2.

In FIG. 2a in particular, it is possible to see that additional quilting seams 8 run between the edge zones 6, each thereby forming a hollow cell 9 together with the seam 7 and the adjacent quilting seams 8. In order for the particular hollow cell 9 to be filled, an opening 16 is worked into the contact layer 4 or the support layer 5, so that rotationally symmetrical massage elements or support elements 10 can be filled into the particular hollow cell 9 through the opening 16 from a holding container 17, for example by means of compressed air. The massage elements or support elements 10 are cylindrical, spherical, oviform, block or pyramid-shaped and have an edge length or a diameter of 1 to 5 mm. If the massage or support elements 10 are filled into the particular hollow cell 9, the massage or support elements 10 create a convexity in the contact layer 4 formed towards the foot 2. The individual hollow cells 9 are aligned in such a way that reflex points of the foot 2 are massaged or, alternatively, the doctor providing the treatment can configure the convexities in the insole 1 in such a way that the foot 2 is orthopedically supported, in order to treat a bone misalignment, for example.

FIG. 2c shows that the support layer 5 is only covered by the contact layer 4 in areas, because the contact layer 4 is absent in the area 20 of the toes of the human foot 2. The support layer 5, on the other hand, covers the entire area of the inside of the shoe 3, with the effect that the edge zone 6 of the support layer 5 is in contact with the inside of the shoe 3. As a result, the support layer 5 serves as the contact surface inside the shoe 3 so that this holds the insole 1 fixed in position inside the shoe 3.

Furthermore, the hollow cells 9 have been individually adapted by means of a bonding seam 19 by the doctor providing treatment to match the orthopedic situation of the patient to be treated. The insoles 1′″ are namely supplied in such a way that only the edge zones 6 of the contact layer 4 and the support layer 5 are connected together by means of the seam 7 or the bonding seam 19.

Once the doctor providing treatment has analyzed the orthopedic situation of the patient, the doctor decides in which areas of the insole 1′″ to arrange the hollow cells 9. Furthermore, the doctor can define what magnitude the convexities of the individual hollow cells 9 should have in order to treat the patient optimally.

In FIGS. 3a and 3b, it is possible to see that a joint hollow cell 9 is enclosed between the contact layer 4 and the support layer 5 into which is inserted a plate 18 matched to the curvature of the foot 2. The one-piece plate 18, that consists for example of plastic, metal or a thermoplastic elastomer material, enables the foot 2 to be orthopedically supported or massaged.

FIG. 4 shows that one or more cushions 11 can be inserted into each of the hollow cells 9, with the cushions 11 possessing a jacket 12. In this case, the jacket 12 encloses an interior into which the massage or support element 10, a gel 13, air 14 or the foam 15 can be filled so that a variation of the described massage or support element can be filled into the corresponding hollow cells 9 of the insole 1 in accordance with the required treatment method.

The openings 16 are resealed using a glue once the hollow cells 9 have been filled in the desired fashion.

All sample embodiments in FIGS. 1 to 4 share the common feature that the materials used for the required components can be sterilized, i.e. exposed to hot steam or pressurized hot steam, without the shape or functions of these components being impaired. In this case, the contact layer 4 is manufactured from a material that can be sterilized and has active breathing properties, in particular linen or synthetic leather and the support layer 5 consists of a thermoplastic elastomer that is unchanged by the steam or pressurized steam treatment. The cushions 11 also have a jacket 12 made from thermoplastic material so that these can also be sterilized by steam or pressurized steam. The fillings provided in the particular cushions 11, namely the massage or support elements 10, the gel 13, the air 14 and the foam 15, are also heat-resistant so that they can be sterilized.

1. An insole (1) for the massage or orthopedic treatment of human feet (2), the insole being arranged in a shoe (3),
the insole (1) comprising a thermoplastic elastomer material which is heat-resistant to such an extent that the insole can be sterilized by a selected one of hot steam and pressurized steam.

2. The insole in accordance with claim 1, wherein,
the insole (1) is formed with a contact layer (4) facing towards a foot (2) and a support layer (5) facing towards the shoe, the contact layer (4) and the support layer (5) being firmly connected to one another in edge zones (6) thereof at least partially around a circumference thereof, by a selected one of a circumferential seam (7), a glue, and a bonding seam (19), and wherein hollow cells (9) are formed in between the contact layer and the support layer by means of quilting seams (8) that separate the hollow cells (9) and wherein one or more of the hollow cells (9) have at least one of massage and support elements (10) inserted therein, or the hollow cells are filled with cushions (11) in which elements (10) comprise at least one of a gel (13), air (14) and foam (14).

3. The insole in accordance with claim 2, wherein
the elements (10) have a rotationally symmetrical shape, and are a selected one of cylindrical, oviform, block-like, and cylindrical in shape, wherein the elements (10) are manufactured from a thermoplastic elastomer.

4. The insole in accordance with claim 2, wherein
the cushions (11) have a jacket (12) into which the elements (10), the gel (13), foam (14) and air (15) are filled, and the jacket (12), and the elements (10), the gel (13) and the foam (14) comprise a thermoplastic material.

5. The insole in accordance with claim 2, wherein
the contact layer (4) comprises a selected one of linen and synthetic leather that can be sterilized and is provided with active breathing properties, and the support layer (5) comprises a thermoplastic elastomer.

6. The insole in accordance with claim 2, wherein
the seam (7), the quilting seams (8) the glues and the bonding seam (19) comprise a heat-resistant material that can be sterilized.

7. The insole in accordance with claim 1, wherein
the insole (1) is formed with a contact layer (4) facing towards a foot (2) and a support layer (5) facing towards the shoe (3), these layers being firmly connected to one another in edge zones (6) thereof by means of a selected one of a seam (7), a bonding seam (19), and a glue, and wherein massage or support elements (10) shaped to match the human foot (2) and formed as a one-piece plate (18) are arranged in between the contact layer (4) and the support layer (5), by means of which the foot (2) is massaged or orthopedically supported.

8. The insole in accordance with claim 7, wherein
the contact layer (4) is formed of a material that can be sterilized and is provided with active breathing properties, and wherein the support layer (5) comprises a thermoplastic elastomer.

9. The insole in accordance with claim 1, wherein
the insole (1) is formed as a selected one of a sock for pulling over a human foot (2), and as an inlay piece adapted to a shape of the shoe (3) for inserting into the shoe (3).

10. The insole in accordance with claim 9, wherein
the sock and the inlay piece each consists of two layers (4, 5), the first layer (4) being in contact with the foot (2) comprises a material that can be sterilized and is provided with active breathing properties, and the second layer (5) facing the shoe (3) being formed from a thermoplastic elastomer.

11. A process for manufacturing a two-layer insole (1) in which one or more hollow cells (9) is or are integrated, the process steps comprising:
working a slit-shaped opening (16) into one of the two layers (4 and/or 5) of the insole (1) so that one or more of the hollow cells (9) can be accessed from outside,
filling a plurality of massage or support elements (10) through each of the worked-in openings (16) and/or,
inserting at least one cushion (11) filled with at least one of the massage or support elements (10), gel (12), foam (14) or air (15), and
closing the opening (16).

12. A process in accordance with claim 11, wherein
the massage or support elements (10) are filled into the hollow cells (9) from a holding container (17) through the openings (16).

13. A process in accordance with claim 11 wherein
each of the openings (16) is automatically closed using a selected one of an adhesive layer, and the material property of the selected layer (4 or 5).
